(19) United States
(12) Patent Application Publication
(10) Pub. No.: US 2009/0155877 A1
(43) Pub. Date: Jun. 18, 2009

(54) Biochip for sorting and lysing biological samples

(75) Inventors: Ciprian Iliescu, Singapore (SG); Guolin Xu, Singapore (SG); Francis Eng-Hock Tay, Singapore (SG)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: Agency for Science Technology and Research, Centros (SG)

(21) Appl. No.: 11/631,667

(22) PCT Filed: Jul. 5, 2005
(86) PCT No.: PCT/SG2005/000220

§ 371 Date: Jan. 5, 2007

Related U.S. Application Data
(60) Provisional application No. 60/586,018, filed on Jul. 6, 2004.

(51) Int. Cl. C12N 13/00; C12M 1/00

(52) U.S. Cl. 435/173.7; 435/306.1; 435/173.9

(57) ABSTRACT

A biochip (100) for lysing and/or cell separation is formed to provide a sealed chamber for biological fluid. A conductive layer (140) bonded between upper (130) and lower (150) insulating layers is etched to form a microfluidic channel (250) between two electrodes (190, 200). The microfluidic channel connects a fluid inlet (11) and fluid outlet (120). The electrodes (190, 200) form an un-even electric field in the channel (250) to generate a dielectrophoretic force on the cells/particles within the sample fluid. A voltage source applies a suitable voltage to separate and/or lyse cells within the fluid.

### CROSS-REFERENCE TO RELATED APPLICATIONS

This application claims benefits from U.S. Provisional Patent Application No. 60/586,718 filed Jul. 6, 2004, the contents of which are hereby incorporated herein by reference.

### FIELD OF THE INVENTION

The present invention relates generally to a biological device for manipulating biological samples and more particularly to a lab-on-a-chip for sorting and lysing biological samples by dielectrophoresis and electroporation.

### BACKGROUND OF THE INVENTION

A growing area of research in the fields of bioengineering and nanotechnology relates to the creation of integrated miniature devices capable of performing chemical and biochemical analysis of biological matter. Such devices are known as micro-total analysis systems (μ-TAS) or a lab-on-a-chip. These devices are becoming increasingly popular in biomedical research, disease diagnosis, food pathogen detection, environmental analysis and forensics. The devices are portable, require reduced sample and reagent sizes, and may be used to conduct point of care testing.

Sample preparation is a critical component and important step in the analysis process carried out by a μ-TAS device. Front-end sample preparation for molecular, biological and immuno-assays generally consists of many steps. After a biological sample such as blood or tissue is obtained for testing, separation and sorting of target cells from the original sample are generally performed. Desired cells that have been isolated from the original sample must also typically be lysed or disrupted in order to release the intracellular components such as DNA and RNA that are to be analyzed. Other steps in front-end sample preparation may include purification of the crude lysate by removing cellular debris, and the amplification of low copy numbers of target nucleic acids.

The use of electrical methods for performing the steps of separating, sorting, and lysing target cells are popular. Two such methods are dielectrophoresis (DEP) and electroporation (EP).

Dielectrophoresis (DEP) is a technique for separating microparticles that has been used for cell separation and sorting. DEP separates particles using dielectrophoretic force, a phenomenon that occurs when a neutral dielectric particle is placed in a varying electrical field that is spatially non-uniform. A dielectrophoretic force is created because the inhomogenous electrical field induces unequal electrical polarization dipoles in the neutral dielectric particle.

The dielectrophoretic force that is experienced by a particle is dependent on a number of factors. The amplitude and frequency of the applied non-uniform electric field will directly affect the dielectrophoretic force experienced by a particle. A particle's own structural and chemical properties will also affect its dielectric properties, and thus its dielectrophoretic movement. For example, a biological cell's morphologies, structural architectures, composition, phenotype, cytoplasmic conductance, cell membrane resistance, capacitance and permittivity, will all affect a cell's polarizability. Also affecting the dielectrophoretic force experienced by a particle are the dielectric properties of the surrounding medium within which the particle is suspended. A particle that is more polarizable than its suspending medium will experience a net force toward high electric field regions (positive DEP), while a particle that is less polarizable than its suspending medium will experience a net force toward low electric field regions (negative DEP). All of these factors affecting the dielectrophoretic force experienced by a particle can be used to exploit the differences between cells of different types or different physiological states such that cell separation and sorting may be performed.

Dielectrophoresis is ordinarily performed on a device having a glass slide with an array of exposed interdigitated and castellated microelectrodes to which an AC voltage is applied. The microelectrode arrays are typically fabricated on the inner surface of the glass plate using a technique such as photolithography. This glass plate generally forms the bottom surface of the DEP separation chamber. Cell mixtures and suspension fluids to be analyzed flow through the microfluidic channel, which is constructed by sandwiching a spacer or gasket with a slot in its centre, between the bottom glass plate and a second glass plate that forms the top of the separation chamber.

As a result of their planar structure, these devices have a number of problems. First, any cells that are located far above the electrode will not move since the electric field strength will be low there. Second, any cells located directly centred and above an electrode will not move because the net force operating on the cell from all electrodes will be zero.

Other general problems associated with conventional dielectrophoresis devices relate to the construction of the flow chamber, microfluidic channels and electrodes. First, these devices require lead-out recesses to connect to an external power supply (off-chip), resulting in fluidic leakage and unwanted local electrical fields within the microfluidic region. Second, unwanted electrochemical effects arise from edge effects in uninsulated multi-metal electrodes such as Ti/Au or Cr/Au. Third, the volume of the flow chamber, (several hundred microliters), is so large that thermal convection exerts unwanted forces on cells.

Electroporation (EP) is another cell preparation technique for creating cell membrane permeability that may be used to perform cell lysis. EP causes cell lysis through the use of pulsed electric fields of high intensity that result in an irreversible breakdown of the cell membrane. This disruption of the bi-layer cell membrane is useful for obtaining intracellular material such as DNA, RNA and mRNA. Empirical evidence has shown that cell membrane properties, the external medium used, and electroporator protocols are all factors that influence effective cell lysis.

Typically, cell separation and lysing steps of sample preparation are performed independently using separate test devices. Such stand alone units require some type of human intervention between the cell separation and lysing steps, leading to increased cost and potential for sample contamination and other operator introduced errors.

In view of the foregoing, a general aim in the development of μ-TAS devices has been to create a small-footprint sample preparation instrument that has the ability to function in an automated and integrated fashion. There continues to exist a need for devices and methods that improve on separation, sorting, and lysing of biological cells.

### SUMMARY OF THE INVENTION

Exemplary of the present invention, a biochip may be used for lysing and/or cell separation. The biochip is formed to provide a sealed chamber for biological fluid. Electrodes are formed therein. The electrodes form an un-even electric field on the channel to generate electric field gradient and hence a force on cells/particle within a fluid in the chamber. A voltage source applies a suitable voltage to separate and/or lyse cells within the fluid.

In accordance with an aspect of the present invention, an apparatus includes a top insulating layer; a bottom insulating layer; and a conductive layer, between the top and bottom insulating layers. The conductive layer is etched to form first and second electrodes separated by at least one microfluidic channel. The top, bottom insulating and conductive layer are bonded together and the microfluidic channel forms a fully sealed flow chamber. An inlet and outlet are in fluid communication with the flow chamber.

In accordance with an aspect of the present invention, a method for cell sorting includes loading a biological sample into a microfluidic channel, formed between two conductive electrodes; applying a potential difference to the conductive electrodes causing target cells to experience a dielectrophoretic force under an electric field; removing unwanted cells from the microfluidic channel using an applied fluidic pressure; and recovering the target cells from the microfluidic channel by removing the electric field and using an applied fluid pressure.

In accordance with yet another aspect of the present invention, there is provided a method for cell lysing, comprising the steps of: loading a biological sample into a microfluidic channel, wherein the microfluidic channel walls are conductive electrodes; applying a potential difference to the conductive electrodes causing target cells to experience a dielectrophoretic force under an electric field moving the target cells to the tips of the conductive electrodes; and applying a high pulse potential difference to the conductive electrodes causing the target cells to experience electroporation.

In accordance with yet a further aspect of the present invention, there is provided a method for performing cell sorting and cell lysing on a single device, comprising: loading a biological sample into a microfluidic channel in the device, wherein the microfluidic channel walls are conductive electrodes; applying a potential difference to the conductive electrodes causing target cells to experience a dielectrophoretic force under an electric field; removing unwanted cells from the microfluidic channel using an applied fluidic pressure; applying a potential difference to the conductive electrodes causing the target cells to experience a dielectrophoretic force under an electric field moving the target cells to the tips of the conductive electrodes; and applying a high pulse potential difference to the conductive electrodes causing the target cells to experience electroporation.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate by way of example only, embodiments of the present invention,

FIG. 1 is a perspective view of a dielectrophoretic biochip exemplary of an embodiment of the present invention;

FIG. 2 is an exploded view of the biochip of FIG. 1;

FIG. 3 is a cross-section view of the biochip of FIG. 1;

FIG. 4 is a bottom plan view of the biochip of FIG. 1;

FIG. 5 is a schematic diagram of a system including the biochip of FIG. 1;

FIGS. 6A to 6F are enlarged schematic diagrams of possible geometric configurations of channel walls of the biochip of FIG. 1 and the electric field vector distribution generated by the electrodes;

FIG. 7 is a graphical illustration of the cell sorting process;

FIG. 8 is a graphical illustration of the cell lysing process;

FIG. 9 is a cross-section view of another embodiment of a dielectrophoretic biochip, exemplary of an embodiment of the present invention; and

FIG. 10 is a bottom plan view of the biochip of FIG. 9.

### DETAILED DESCRIPTION

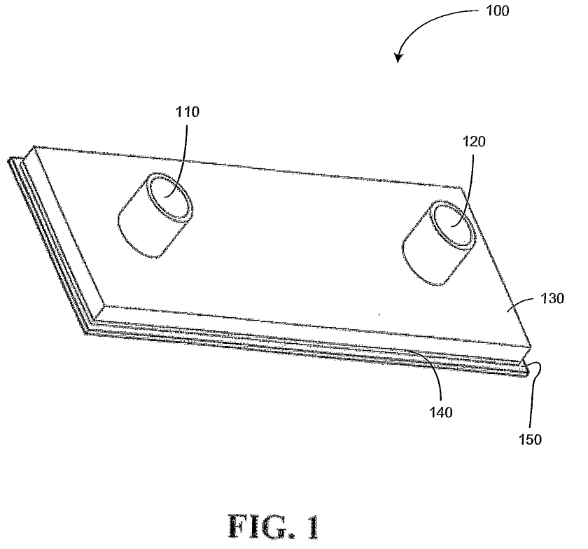

FIGS. 1 to 4 illustrate a device in the form of a biochip 100 exemplary of an embodiment of the present invention. As will become apparent, biochip 100 may be used for dielectrophoretic separation of biological material and lysing.

As illustrated in FIG. 1, biochip 100 includes an inlet 110, an outlet 120, top, middle and bottom layers 130, 140 and 150. Top layer 130 and bottom layer 150 are each formed as insulating wafers, formed for example composed of a material such as glass. Middle layer 140 is formed of a conductive material, such as for example silicon or metal.

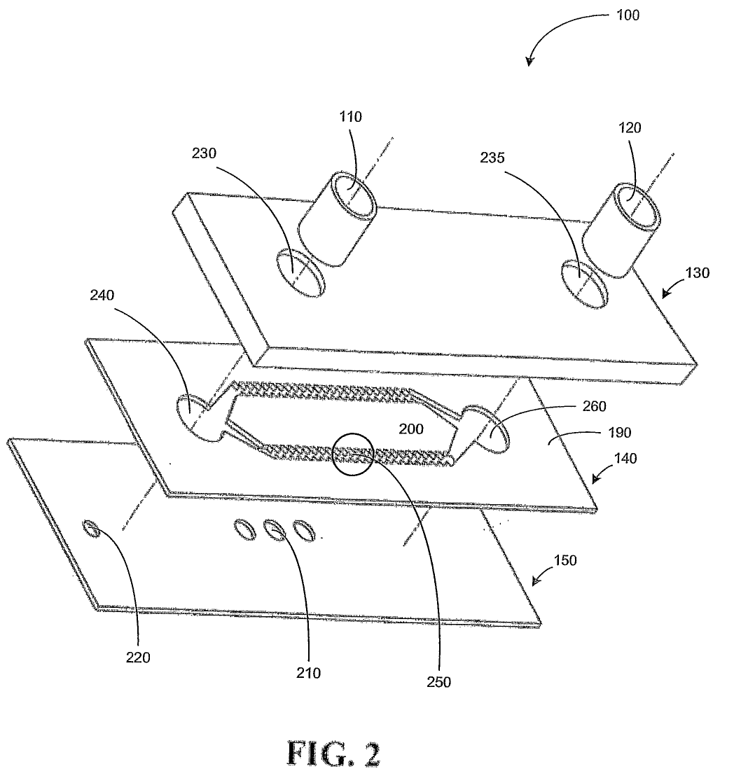

As illustrated in FIG. 2, a central portion of layer 140 forms a central electrode 200 and may be formed using etching or electroforming. The remainder of layer 140 acts as a bulk electrode 190. All three layers 130, 140, 150 are assembled to create a completely enclosed flow chamber defined by microfluidic channels 250. Top layer 130 and bottom layer 150 form a ceiling and floor of the flow chamber.

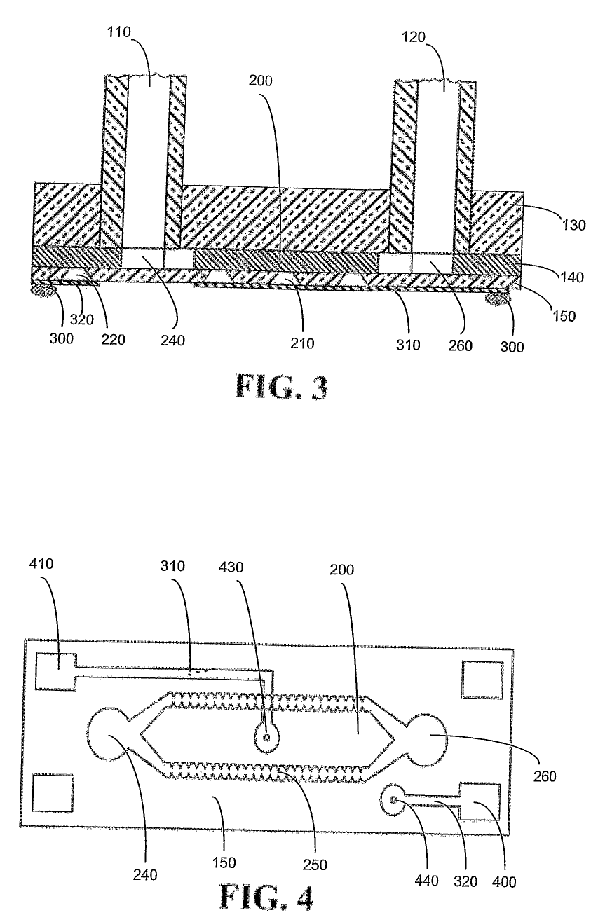

Metalized vias 210, 220 illustrated in FIGS. 2 and 3 extend through bottom layer 150 to provide both mechanical and electrical connection to central electrode 200 and bulk electrode 190. Metallization strips 310, 320 visible in FIGS. 3 and 4, formed on the bottom of bottom layer 150 electrically connect central electrode 200 to voltage pad 410 and bulk electrode 190 to voltage pad 400. Voltage pads 400, 410 are electrically connected to solder bumps 300 (FIG. 3). Pads 430, 440 interconnect strips 310, 320 to vias 210, 220. Solder bumps 300 provide external electrical connections to the electrodes 190, 200 and allow biochip 100 to be connected to a printed circuit board (PCB) that may supply the biochip 100 with applied voltage.

Inlet 110 is in flow communication with entrance 240 to channels 250 through inlet hole 230 that is drilled on the top of glass wafer forming top layer 130. Outlet 120 is similarly connected to an exit 260 of channels 250 through a second hole 235 in the top glass wafer forming layer 130. The inlet/outlets 110/120 allow the loading and unloading of biological samples in channels 250. Example approximate dimensions of biochip 100 are 12 mm (length), 4 mm (width) and 1.1 mm (height). Electrodes 190, 200 range in thickness of approximately 50-700 μm. The size of microchannels 250 ranges approximately from 20-500 μm in distance between electrodes 190 and 200.

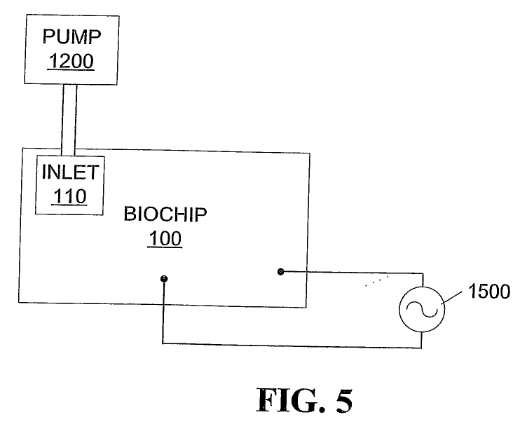

As schematically illustrated in FIG. 5, biochip 100 may be interconnected with a pump 1200 and a voltage source 1500. Exemplary voltage source 1500 is an AC function generator. Pump 1200 is in flow communication with inlet 110 to urge a biological or similar fluid into channels 250. Function generator 1500 is interconnected with solder bumps 300 to provide a time varying voltage across electrodes 190, 200 as detailed below. The applied frequency of function generator 1500 may range from approximately 1 KHz to approximately 100 MHz with peak to peak voltage may range from approximately 5Vₚ₋ₚ to approximately 50Vₚ₋ₚ.

The small dimensions of the flow chamber and microfluidic channels 250 allow for a small working volume of approximately 1 μL. Moreover, relatively low voltage between electrode 200 and bulk electrode 190 results in a strong electric field across microfluidic channels 250. The shape and dimensions of channel 250, and particularly, the side walls defining channel 250 affect the nature of the field in channel 250.

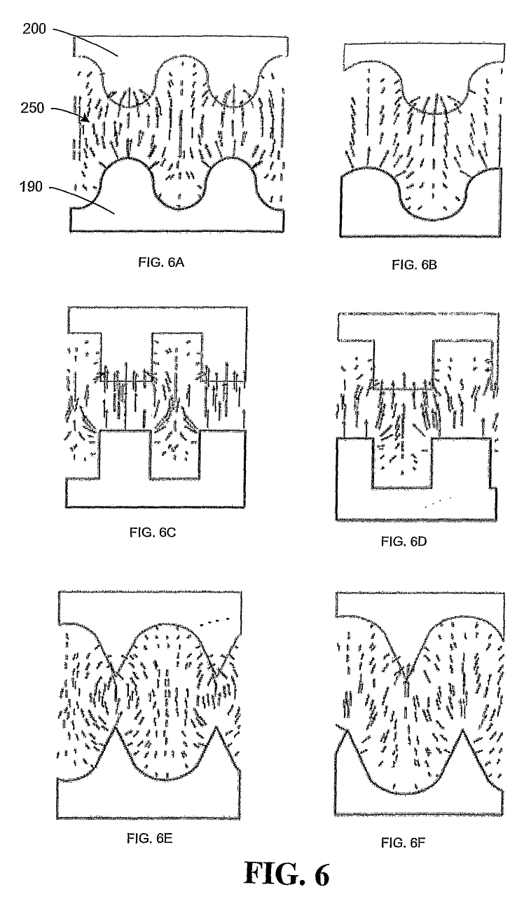

FIGS. 6A to 6F accordingly illustrate possible wall shapes and configurations of the side walls of channel 250 (as defined by the shape of bulk electrode 190 and central electrode 200). Electric field vectors of the configurations are also illustrated. Numerous other wall shapes are possible, and will be apparent to those of ordinary skill.

FIGS. 6A and 6B illustrate generally semi-circular electrode tips with opposite (FIG. 6A) and offset (FIG. 6B) configurations. The radius of the semi-circle is approximately 50 μm-400 μm.

FIGS. 6C and 6D illustrate an interdigitated electrode tips with opposite (FIG. 6C) and offset FIG. 6D configurations. The dimensions of the interdigitated electrode tips are generally rectangular and approximately 50 μm-400 μm in width and 50 μm-400 μm in length.

FIGS. 6E and 6F depict an example triangular electrode structure with opposite (FIG. 6E) and offset (FIG. 6F) configurations. The angle of the triangle forming the tips of the electrode ranges approximately from 30-90 degrees. The height of the triangle is in the approximate range of 50 μm-200 μm and the diameter of the arc forming the bays between two adjacent tips is in the approximate range of 50 μm-200 μm.

The strongest electric field and its gradient are located in the areas where the space between bulk electrode 190 and central electrode 200 is the smallest. These are indicated in FIGS. 6A to 6F by arrows of longer length. Arrows of smaller length indicate locations where the electric field and its gradient are the weakest (such as in the bays in between two adjacent tips of an electrode). Of the three electrode shape configurations illustrated in FIGS. 6A and 6B; 6C and 6D; and 6E and 6F, the electric field strength is greatest for the triangular electrode tips of FIGS. 6E and 6F and the weakest for the generally rectangular electrode tips of FIGS. 6C and 6D (assuming the applied voltage and distance between bulk electrode 190 and central electrode 200 is the same for all configurations).

For example, using the triangular wall shape configuration depicted in FIGS. 6E and 6F, electric field strengths higher than 1.6×10⁵ Vm⁻¹ will be achieved at a distance of 10 μm from the electrode tips by using a small operating voltage of 25Vₚ₋ₚ. Very small spacing (less than 100 μm) between triangular wall tips of central electrode 200 and bulk electrode 190 results in such field strengths.

The glass-silicon-glass structure of example biochip 100 may be fabricated using wafer level anodic bonding to create a fully sealed fluidic environment as follows. Other materials and fabrication techniques will be readily apparent to those of ordinary skill.

First, top glass wafer forming layer 130 with inlet/outlet holes 230/235 is fabricated. A four-inch glass wafer (Pyrex glass Corning 7740) with a thickness of 700 μm, for example, may be drilled using diamond bits to create inlet hole 230 and outlet hole 235. The holes are of a diameter of approximately 1.6 mm each.

Next, the top glass layer 130 is anodically bonded to the silicon wafer defining layer 140.

Thirdly, electrodes 200, 190 and microfluidic channels 250 are etched in layer 140 using a deep reactive ion etching (RIE). For this step, a photoresist mask may be used. The deep RIE etching is done through the silicon wafer 140 with a stop-etch on the glass substrate

After removal of the photoresist layer, a further wafer to wafer bond is made. The wafers are prepared in a similar manner to the first bonding step. The alignment between the glass wafers 130, 150 and the silicon electrodes 200, 190 is performed manually on the bonding frame of a wafer bonder.

Next, the bottom glass wafer forming layer 150 is thinned in order to simplify connection of the electrodes 200, 190 using vias 210, 220, respectively, that are formed through the bottom layer 150. A wet etching process for creating a 500 μm deep hole from one side is not yet reported in the literature. After a deep wet etching process the size of generated holes becomes very large due to the isotropy of etching (for a 500 μm thick wafer the final diameter is greater than 1 mm). For this reason, it is necessary to make thin the bottom glass wafer 150. This process was performed by wet etching in an optimized HF (49%)/HCl (37%) 10/1 solution. The glass used (Corning 7740) offers good bonding performance on silicon as well as a low content of oxides that gives rise to insoluble products in HF (e.g. CaO, MgO or Al2O3). These insoluble products can increase the roughness of the surface due to their re-deposition during the wet etching process. The role of HCl in the solution is to transform the insoluble products into soluble products.

The presence of insoluble products can drastically reduce the etching rate. The thinning process is performed using a Teflon beaker and slow magnetic stirring until the thickness of the glass is around 100 μm. The uniformity of the process is relatively good with only 20-25 μm thickness variation after 400 μm of etching (the process uniformity is around 5%). Temporarily bonding a protective silicon wafer with wax assures the protection of the other front side of the processed wafer, during the wet etching.

Next, via-holes 210, 220 are formed in the bottom glass wafer of layer 150. An ACr/Au (50 nm/1 μm) masking layer is used for the via-holes 210, 220. The patterning of the Cr/Au was performed using a 2 μm thick photoresist (for example, AZ7220) and classical gold and chromium etchants. The photoresist mask is hard baked at 135° C. for 30 minutes in order to improve the adhesion of photoresist on the Au layer. During the etching process the photoresist layer reduces the likelihood of cracking bottom layer 150. The tensile stress induced in the Cr/Au masking layer can lead to cracks in the mask and the highly concentrated HF solution can penetrate through these cracks and generate pinholes. Although the resistance of photoresist in HF etching solution is poor, the photoresist layer can still improve the overall etching resistance of the masking layer. During the spin-coating process the photoresist penetrates and fills cracks. Hard-baking the photoresist increases its adhesion on surfaces and removal of photoresist from the cracks during the wet etching process becomes more difficult.

The thickness of the gold layer also plays an important role in a good etching process. After the wet thinning of the wafer, the surface roughness (Rₐ) increases from 1 nm up to 10 nm. A large value of surface roughness will increase the number of stress concentration points in the masking layer and therefore increase the number of cracks. By increasing the thickness of the Au layer, the penetration of the etching solution through the cracks (nano-channels) is slower so the overall performance of the masking process is improved. Etching vias 210, 220 is performed in the same solution: HF/HCl 10/1. The Cr/Au mask is removed in the same Cr and Au etchants. Another wet etching process for 1.5 min (equivalent to a depth etch of 10 μm) is performed mainly to remove the sharpness of the edges and also to remove the non-uniform effects of the wet etching process (during the via-holes etching the etch-stop process cannot be realized due to the small size of the mask and the large depth of etching). The modification of the edge sharpness plays an important role for the subsequent steps of the fabrication process: the photoresist flow during the spincoating is more uniform, and the risk of metallization stepcoverage issues over a sharp edge is eliminated.

Metallization leads 310, 320 are formed on the bottom of layer 150. Metallization may be performed using a Cr/Au (50 nm/1 μm) layer deposited using an e-beam evaporator. A thick positive photoresist (for example, AZ9260) may be employed to define the layer. For better uniformity, the spinning of the photoresist is performed at a low speed (1500 rpm). To increase the coverage of the vias, the thickness of photoresist may be increased by performing a double coating. The final thickness of the photoresist (measured on a planar surface) is 24 μm. The mask is overexposed in order to cover the non-uniformities of the deposited photoresist. After photoresist patterning, the Cr/Au layer is etched using a similar process to that used for the Cr/Au etching mask.

Conventional micromachining fabrication processes may be employed for the fabrication of biochip 100. For example, electroplating may be used when the electrode material is metal.

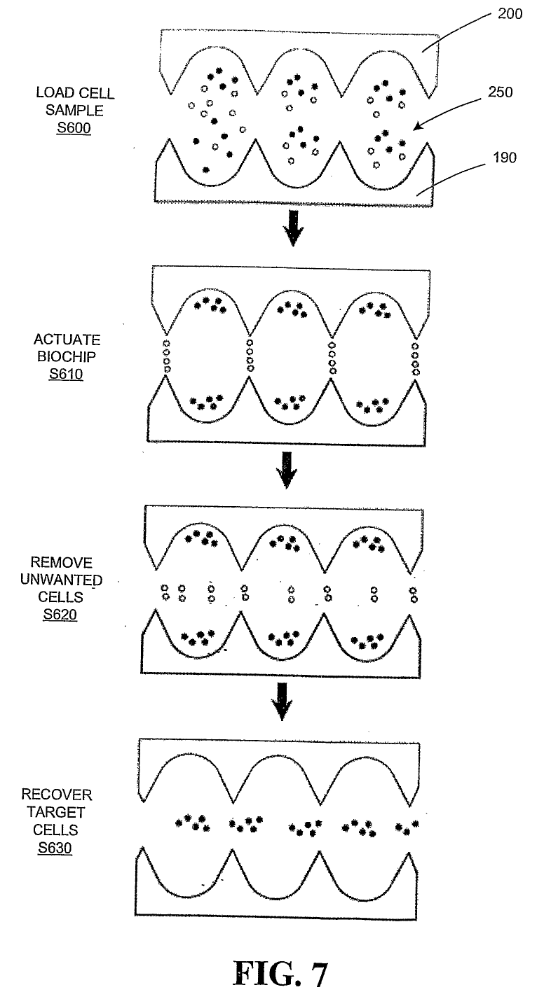

FIG. 7 is a graphical illustration of exemplary steps of operating the biochip 100 for cell sorting by dielectrophoresis. The four illustrations shown in the FIG. 7 are close-up views of a microfluidic channel 250, having the shape depicted in FIG. 6E-6F. The depicted black particles represent target cells and the white particles represent non-target cells. The general steps involved in selectively sorting target cells from unwanted cells include loading a cell sample, actuating the biochip, removing unwanted cells, and recovering target cells.

As illustrated, the biological sample that is to be processed by the device 100 is first loaded in step S600 into device 100. Many different particles, including the target cells of interest, will be suspended in the fluid that is loaded into biochip 100. Pump 1200 (FIG. 5) may be employed to introduce the sample into the microfluidic channel 250 by way of the inlet 110 and flow chamber entrance 240. For example, pump 1200 may be a syringe pump used to accomplish cell-loading step S600. Other similar instruments familiar to a person skilled in the art may be used. These might include peristaltic pumps, pipettors, and Hamilton syringes. The loading of the cell sample in the biochip device 100 can be entirely automatic or manual depending on the device user's requirements.

Device 100 is actuated in step S610. FIG. 7 illustrates the physical separation of target cells (shown as black particles) and unwanted cells (shown as white particles) that results from activation of the biochip 100. Dielectrophoresis separates the particles using dielectrophoretic forces that are experienced by the particles. These forces are generated through the application of a non-uniform electric field across the microfluidic channel 250. solder bumps 300 of the biochip 100 may be electrically connected to a printed circuit board (PCB), thus allowing the non-uniform electric field to be created by a voltage source 1500. The function generator 1500 outputs a waveform (e.g. a sinusoid) to generate the AC drive signal in order to drive the biochip 100.

Dielectrophoresis (DEP) separates particles using dielectrophoretic force, a phenomenon that occurs when a neutral dielectric particle is placed in an electrical field that is spatially non-uniform. A dielectrophoretic force is created because the inhomogenous electrical field induces unequal electrical polarization dipoles in the neutral dielectric particle.

Biological cells may be modeled as spherical particles. The DEP force acting on a cell with a radius of r, can be determined using the following equation:

F=2πr³∈ₘRₑ[K]∇E²

where ∈ₘ is the absolute permittivity of the suspending medium, ∇E is the local electric field (rms) intensity, and Rₑ[K] is the real part of the polarization factor (Clausius-Mossotti factor). The polarization factor is defined as:

$$K = \frac{{ɛ}_{p}^{*} - {ɛ}_{m}^{*}}{{ɛ}_{p}^{*} + 2 {ɛ}_{m}^{*}}$$

where ∈_{P}* and ∈ₘ* are the complex permittivity of the particle and medium respectively. The complex permittivity for a dielectric material can be described by its permittivity \grave{∈} and conductivity σ, where ω is the angle frequency of the applied electrical field E:

$${ɛ}^{*} = ɛ - j \frac{σ}{ω}$$

The above equations demonstrate that the DEP force that is experienced by a particle is dependent on a number of factors. That is, the DEP force is proportional to the volume of the particle and ∈ₘ, the dielectric permittivity of the medium in which the particle is suspended. It is also seen that particles will only experience a DEP force when the electric field is non-uniform and that the force does not depend on the polarity of the electric field, (allowing AC excitation to be used).

From the Clausius-Mossotti function, the DEP force is shown to depend on the magnitude and sign of the real part of the effective polarization factor K. Thus, both the amplitude and frequency of the applied alternating current (AC) non-uniform electric field will directly affect the dielectrophoretic force experienced by a particle. A particle's own structural and chemical properties will also affect its dielectric properties, and thus its dielectrophoretic movement. For example, a biological cell's morphologies, structural architectures, composition, phenotype, cytoplasmic conductance, cell membrane resistance, capacitance and permittivity, will all affect a cell's polarizability.

Also affecting the dielectrophoretic force experienced by a particle are the dielectric properties of the surrounding medium within which the particle is suspended. The effect that the relationship between the dielectric properties of the particle and the suspending medium has on the DEP force is described by the Clausius-Mossotti function. If Rₑ[K] is a positive value, then:

∈ₚ>∈ₘ

This means that a particle is more polarizable than its suspending medium and as a result will experience a net force toward high electric field regions due to an induced dipole moment aligned with the applied field, a phenomenon known as positive dielectrophoresis. When a particle is less polarizable than its suspending medium, then Rₑ[K] is a negative value and:

∈ₚ<∈ₘ

Under these conditions, negative dielectrophoresis is the result, and the particle experiences a net force toward low electric field regions, due to an induced dipole moment aligned opposite to the applied field. The theoretical bounds for the real part of K are:

−0.5<Rₑ[K]<+1.0

As noted, the nature of the electric field created across microfluidic channel 250 is also strongly affected by the specific physical configuration and structure of the electrodes. For example, field concentrations are greatest at the sharp edges of the electrodes and where electrodes of opposing polarity are closest to each other, as illustrated in FIGS. 6A to 6F. The DEP force will typically reduce exponentially as the distance from the surface of the electrode increases. DEP force also decays as a function of L⁻³, where L is a characteristic length of an electrode. These relationships demonstrate that the geometric size and shape of electrodes influence the applied electric field.

All of these factors that affect the DEP force experienced by a particle can be used to exploit the differences between cells of different types or different physiological states such that the cells will separate when the device is actuated in step S610. By selecting appropriate parameters, the target cell DEP characteristics can be used to physically separate the target cells from other unwanted cells.

The selection of appropriate parameters will depend on both the characteristics of the particular cells to be sorted and the geometry of the electrodes on the device, but will be apparent to a person skilled in the art. For example, if a positive DEP causing target cells to move toward high electric field regions is desired, then Rₑ[K] should be a positive value. This means that a suitable suspending medium is selected such that its relative permittivity is lower than that of the target cells. A suitable frequency of the applied electric field must also be selected, since the AC frequency of the applied field also directly affects the polarity of Rₑ[K]. For example, the higher the conductivity of the suspending medium, the higher the applied voltage frequency is required in order to induce positive DEP. Based on the positive and negative DEP forces, the cells in the biological sample can be separated or concentrated.

For example, the operation of biochip 100 for the sorting of mammalian red blood cells may include the suspension of red blood cells in a 8.5% sucrose and 0.3% dextrose suspension fluid. The conductivity of the fluid may be adjusted to 20-100 mS/m with a phosphate buffered saline (PBS) buffer. If the suspending medium has a conductivity of 32 mS/m, red blood cells will exhibit negative DEP if the applied frequency is lower than 119 KHz, (and the cell will move to areas where the electric field is the weakest). If the applied frequency is higher than 119 KHz, the cells will exhibit positive DEP and they will move to areas where the electric field is the strongest. If the conductivity of the suspending medium is increased to 92 mS/m, then an applied frequency of less than 387 kHz will induce negative DEP in the cells and an applied frequency of greater than 387 kHz will induce positive DEP in the cells.

As will be appreciated, prior to introduction of the biological sample into microchannel 250, the DEP characteristics of the cell sample of interest in the biological sample are typically studied so as to choose an appropriate conductivity suspending medium and an appropriate actuation frequency of the voltage source 1500.

As shown in step S610 of FIG. 7, when the device is actuated an AC current is applied to the electrodes 190, 200 creating a non-uniform electric field across the microfluidic channel 250, in a first mode to separate cells in the biological fluid. The physical separation of target cells (shown as black particles) and unwanted cells (shown as white particles) is a result of the dielectrophoretic forces acting on the cells. In this particular example, the unwanted cells are moved to the electrode 190, 200 edges or tips where electric field concentration is the highest, while the desired cells are moved to bay regions between the neighboring electrode 190, 200 castellation tips Where the electric field concentration is the lowest.

In step S620, unwanted cells are removed from the microfluidic channel 250 to the flow chamber exit 260 and unloaded from the biochip 100 via the outlet tube 120. Pump 1200 that is used to load the biochip 100 with the cell sample may also be used to provide a method of fluid transport out of the biochip 100. For example, a syringe pump 1200 may be used to continuously pump biological samples into the microfluidic channel 250, while simultaneously removing unwanted cells. If DEP forces keep unwanted cells in the center of the channel and target cells in the bay regions between castellated tips, only the unwanted cells will be forced out of the channel 250 along with the suspending fluid.

In step S630, the target cells are recovered. Once the cell sorting has been completed on the entire input sample, the target cells may be obtained by turning off the actuation voltage. This releases the cells from the bay regions, allowing the desired cells to be forced out of the channel 250 and the outlet 120 under the fluidic pressure created by the syringe pump 1200. A phosphate buffered saline (PBS) solution may be used as a re-suspension fluid, for the purpose of removing the target cells.

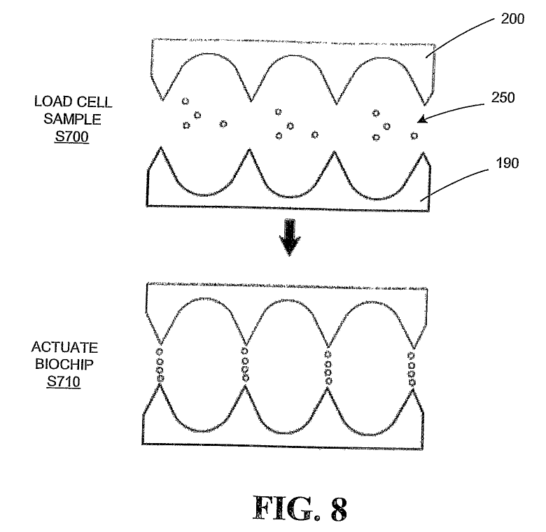

FIG. 8 is a graphical illustration of an exemplary process of operating the biochip 100 for cell lysing by electroporation. Optionally, DEP and electroporation may be performed on the sample in sequence. Alternatively, biochip 100 may be used to perform either DEP or electroporation, on individual samples. As will become apparent, the mode of operation of biochip 100 varies with the voltage applied to electrodes 190 and 200, by way of voltage source 1500.

The two illustrations shown in the FIG. 8 are close up views of a microfluidic channel 250 that is defined by a central electrode 200 and a bulk electrode 190. The white particles represent cells that are selectively lysed in this process. The general steps involved in lysing cells include loading a cell sample, actuating the biochip, and capturing desired intracellular material.

So, as illustrated in FIG. 8, the biological sample that is to be processed by the device 100 is first loaded into the device 100. Pump 1200 may be employed to introduce the sample into the microfluidic channel 250 by way of the inlet 110 and flow chamber entrance 240. For example, a syringe pump 1200 may be used to accomplish cell-loading step S700. Other similar instruments familiar to a person skilled in the art may be used. These might include peristaltic pumps, pipettors, and Hamilton syringes. The loading of the cell sample in the biochip device 100 can be entirely automatic or manual depending on the device user's requirements. It is also contemplated that after successful cell sorting using DEP, electroporation may subsequently be performed, thus allowing cell separation and lysing to be executed with the same biochip device 100.

Biochip 100 is actuated in step S710 in its second mode. To lyse or disrupt the cells in the biological sample, a low voltage is first applied to create a DEP force, so that the cells move to the tips of the electrodes 190, 200. This portion of step S710 is similar to step S610 in the cell sorting process and the selection of appropriate system parameters needed to move the cells to the tips of the electrodes 200, 190 will be apparent to a person skilled in the art.

After the cells have been collected at the electrode 190, 200 tips, a high pulse voltage is applied in order to lyse or disrupt the cells causing an irreversible breakdown of the cell membrane. For many types of cells, lysis will occur when the trans-membrane potential (TMP) that is induced across the cell membrane is raised to about 1V. Empirical evidence has shown that cell membrane properties, the external medium used and electroporator protocols are all factors that influence effective cell lysis.

For biological cells that may be modeled as spherical particles, a time-independent expression of the trans-membrane potential ∇φ induced by an external electric field is:

∇Φ=1.5rE cos θ

where r is the cell radius, E is the density of the external electric field, and θ is the angle between the field line and the normal point of interest in the cell membrane.

The trans-membrane potential creates micro-pores on the cell membrane, so that intracellular material such as DNA, RNA and mRNA can be moved into or out of the cell by electro-osmosis and diffusion. A smaller size of cell diameter requires a higher electrical field E to generate the same trans-membrane potential. After the cell has been lysed, the intracellular material released by the cell may be captured using a variety of methods. For example, beads coated with a magnetic surface, such as Oligo (dT), may be used to capture mRNA. The same pump 1200 that is used to load the biochip 100 with the cell sample may be used to provide a method of fluid transport out of the biochip 100 in order to remove the desired intracellular material from the microfluidic channel 250.

The biochip device 100 has been successfully applied to the DEP manipulation of yeast cells. 100 mg of yeast, 100 mg of sugar and 2 ml DI water were incubated in an Eppendorf tube at 37° C. for 2 h. The cells were then concentrated by centrifugation at 1000 rpm for 1 minute. The supernatant solution was removed and the cell pellet was washed by adding 2 ml of DI water into the tube. The centrifugation and washing process was repeated three times before the cells were collected and resuspended in the separation buffer, which was a mixture of phosphate buffered saline (PBS) and DI water. The conductivity of the separation buffer was adjusted to about 50 μS cm-1 using NaOH. The final concentration of the cells was 1×10⁷ cell ml-1.

Prior to injecting the cell suspension into the device 100, the protocol was to assure that the voltage source 1500 and associated amplifier were powered on but their outputs were set to the minimum. This was to prevent the generation of bubbles inside the channel 250 by electrolysis. 1 μl of the cell mixture suspension was injected into the biochip 100. The drive signal was increased from 0 to 25 Vₚ₋ₚ gradually. The signal frequency was anywhere in the range of 20 kHz to 100 kHz. When the actuation signal reached 13 Vₚ₋ₚ, cells began to move towards the tip of electrodes 190, 200 where the electric field gradient was highest. As the actuation voltage increased, the cells moved faster. For a 25 Vₚ₋ₚ voltage, a stable equilibrium cell concentration pattern was achieved in 10-13 seconds.

Exemplary device 100 was free from electrical dead volumes. The majority of the cells were concentrated in the strongest electric field region around the electrode 200, 190 tips. Since the cells are concentrated together in the highest electric field area, they can also be easily lysed. In a typical device, particles directly above the 2D electrodes do not experience a useful DEP force and cannot be manipulated. Conveniently, device 100 may also be free from fluidic dead volumes. This was verified by priming the device 100 with cells and counting to confirm that all the cells were immediately flushed out when no DEP voltage was applied.

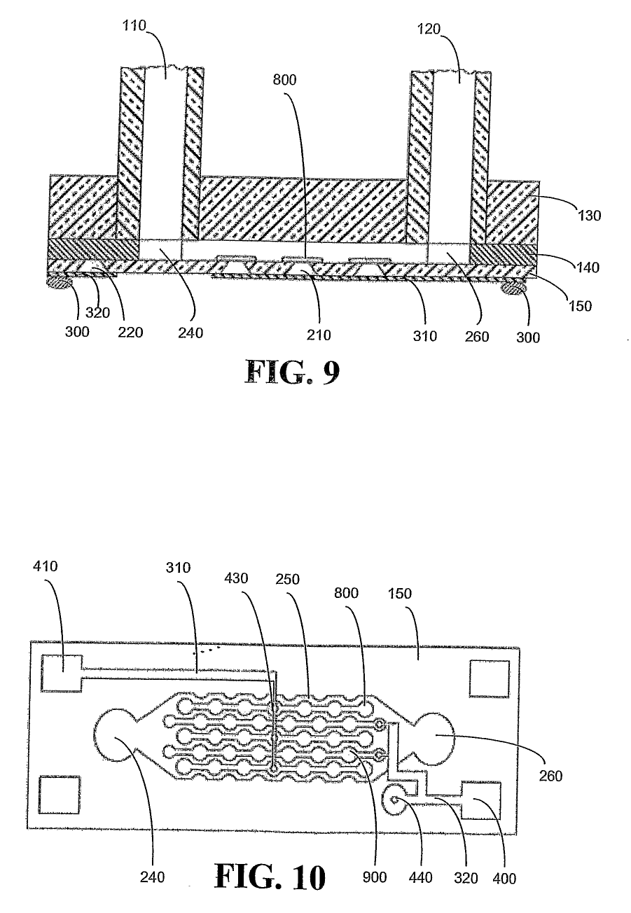

A second exemplary biochip device 100′ is illustrated in FIGS. 9 and 10. In this embodiment a DEP device 100′ with a variable force in the Z-direction is presented. A strong and even electric field in Z-axis is created using an electrode configuration including a thick bulk electrode 900, shown in FIG. 10, and thin circular electrodes 800, shown in FIG. 9. The thin circular electrodes 800 are created using doped amorphous silicon and are approximately 700 nm in thickness. The thick bulk electrode is approximately 100 μm in thickness.

Due to the large difference in thickness between the thin electrodes 800 and the thick bulk electrode 900, a force gradient in the Z-direction is assured in addition to the force gradient in the XY plane. Microfluidic channels 250 are defined by the walls of the thick bulk electrode 900 and numerous thin electrodes 800 may be patterned on the bottom glass wafer 150 within these channels.

The specific physical configuration and structure of thick bulk electrode 900 and thin electrodes 800 affect the nature and strength of the electric field. For example, using circular shaped thin electrodes 800 having a diameter of 50-200 μm and a height of 0.1-1 μm spaced very closely (less than 100 μm) to a thick bulk electrode 900 having a height of 100-500 μm achieves a strong electric field across microfluidic channels 250 using a low operating voltage.

Biochip 100′ may be fabricated in a manner similar to biochip 100.

Of course, the above described embodiments, are intended to be illustrative only and in no way limiting. The described embodiments of carrying out the invention, are susceptible to many modifications of form, arrangement of parts, details and order of operation. The invention, rather, is intended to encompass all such modification within its scope, as defined by the claims.

1. An apparatus, comprising,
a top insulating layer;
a bottom insulating layer;
a conductive layer, between said top and bottom insulating layers, said conductive layer etched to form first and second electrodes separated by at least one microfluidic channel;
wherein said top, bottom insulating and conductive layers are bonded together and said at least one microfluidic channel forms a fully sealed flow chamber;
an inlet in fluid communication with said flow chamber; and
an outlet in fluid communication with said flow chamber.

2. The apparatus of claim 1, further comprising metalized vias through said bottom insulating wafer, interconnected to said electrodes.

3. The apparatus of claim 2, further comprising solder bumps on said bottom insulating layer connected with said metalized vias.

4. The apparatus of claim 1, wherein said top insulating layer and said bottom insulating layer are formed of glass.

5. The apparatus of claim 1, wherein said conductive layer is formed of doped silicon.

6. The apparatus of claim 1, further comprising a voltage source interconnected with said electrodes, to apply a time varying potential difference across said electrodes and said microfluidic channel.

7. The apparatus of claim 6, wherein said voltage source is operable to control said potential difference waveform output in a first mode for applying a dielectrophoretic force to cells within said fluid to sort cells within said fluid.

8. The apparatus of claim 7, wherein said voltage source is operable to control said potential difference waveform output in a second mode to lyse cells in said biological fluid.

9. The apparatus of claim 8, further comprising a pump in fluid communication with said inlet and said outlet to create fluid pressure within said flow chamber.

10. The apparatus of claim 1, comprising a third electrode disposed in said channel between said first and second electrodes, wherein said first electrode comprises a bulk electrode and said third electrode is thinner than said bulk electrode.

11. The apparatus of claim 1, wherein said first and second electrodes include generally rectangular tips with dimensions of between 50 to 400 μm by 50 to 400 μm.

12. The apparatus of claim 1, wherein said first and second electrodes include generally semi-circular tips.

13. The apparatus of claim 12, wherein each of said semi-circular tips has a radius of between about 50 and 400 μm.

14. The apparatus of claim 11, wherein said tips of said first electrode are interdigitated with said tips of said second electrode.

15. The apparatus of claim 1, wherein said first and second electrodes each have a thickness between about 50 μm and about 700 μm.

16. The apparatus of claim 1, wherein the width of said at least one microfluidic channel between said first and second electrode is between about 20 micrometres and 500 micrometres.

17. The apparatus of claim 1, wherein said first and second electrodes have tips that are generally triangular in shape.

18. The apparatus of claim 10, wherein said bulk electrode is about 100 μm in thickness and said third electrode is about 700 nm in thickness.

19. A method for cell sorting, comprising:
loading a biological sample into a microfluidic channel, formed between two conductive electrodes, said electrodes comprising tip portions and thus defining a center region and bay regions in said channel;
applying a potential difference to said conductive electrodes causing target cells to experience a dielectrophoretic force under an electric field and thus move into said bay regions, and keeping unwanted cells in said center region;
removing said unwanted cells from said microfluidic channel through said center region using an applied fluidic pressure; and
recovering said target cells from said microfluidic channel through said center region by removing said electric field and using an applied fluid pressure.

20. The method of claim 19, wherein said dielectrophoretic force exists in three dimensions.

21. A method for cell lysing, comprising:
loading a biological sample into a microfluidic channel, wherein said microfluidic channel walls are conductive electrodes;
applying a potential difference to said conductive electrodes causing target cells to experience a dielectrophoretic force under an electric field moving said target cells to the tips of said conductive electrodes; and
applying a high pulse potential difference to said conductive electrodes causing said target cells to experience electroporation.

22. The method of claim 21, further comprising recovering intracellular material from said target cells using beads coated with a magnetic surface.

23. The method of claim 22, wherein said dielectrophoretic force exists in three dimensions.

24. A method for performing cell sorting and cell lysing on a single device, comprising:
loading a biological sample into a microfluidic channel in said device, wherein said microfluidic channel walls are conductive electrodes;
applying a potential difference to said conductive electrodes causing target cells to experience a dielectrophoretic force under an electric field;
removing unwanted cells from said microfluidic channel using an applied fluidic pressure;
applying a potential difference to said conductive electrodes causing said target cells to experience a dielectrophoretic force under an electric field moving said target cells to the tips of said conductive electrodes; and
applying a high pulse potential difference to said conductive electrodes causing said target cells to experience electroporation.

25. The method of claim 24, further comprising recovering intracellular material from said target cells using beads coated with a magnetic surface.

26. The method of claim 25, wherein said dielectrophoretic force exists in three dimensions.
